(19) United States
(12) Patent Application Publication
(10) Pub. No.: US 2009/0022811 A1
(43) Pub. Date: Jan. 22, 2009

(54) Mineralized guided bone regeneration membranes and methods of making the same

(76) Inventors: Racquel Z. LeGeros, New York, NY (US); Hung-Kuo Chou, New York, NY (US); Dindo Mijares, Woodside, NY (US); John P. LeGeros, New York, NY (US)

Correspondence Address: KLAUBER & JACKSON, 411 HACKENSACK AVENUE, HACKENSACK, NJ, 07601, US

(21) Appl. No.: 12/075,124

(22) Filed: Mar. 7, 2008

Related U.S. Application Data
(60) Provisional application No. 60/905,516, filed on Mar. 7, 2007.

(51) Int. Cl. A61K 33/42; A61P 19/00; A61F 2/00

(52) U.S. Cl. 424/602; 427/2.26; 424/601; 424/425

(57) ABSTRACT

The present invention provides a method for mineralizing commercially available guided bone regeneration membranes. The method comprises the steps of (a) providing a commercially available guided bone regeneration membrane, (b) applying a mineralizing solution, and (c) microwaving the membrane. The method may further comprise (d) rinsing the membrane in a solution such as distilled water and (e) drying the membrane. The mineralizing solution may be a solution capable of supplying or delivering a mineral such as calcium or zinc. The invention further provides guided bone regeneration membranes made by the methods described. The guided bone regeneration membrane comprises a mineral, such as, for instance calcium or zinc at a weight percent of at least 5%, at least 10%, at least 12%, at least 15%, at least 18%, at least 20% or at least 25% (weight percent) of the membrane. Further, the invention provides methods for enhancing bone regeneration and methods for inhibiting bacterial infection and inflammation.

### FIELD OF THE INVENTION

This invention relates to mineralized guided bone regeneration membranes and methods of making the same.

### BACKGROUND

Guided bone regeneration (GBR) or guided tissue regeneration (GTR) membranes are used as biological and mechanical barriers to prevent fibroblasts and other soft connective-tissue cells to migrate into the defect sites or bone grafted sites, and allow only the slow-migrating osteogenic cells to occupy and repopulate the defect sites (Dahlin et al., Plastic & Reconstructive Surgery 1988; 81(5):672-676; Simion M, Scarano A, Gionso L, Piattelli A. Guided bone regeneration using resorbable and nonresorbable membranes: A comparative histologic study in-humans. Int J Oral Maxillofac Implant. 1996; 11:735-742). The membranes serve as biological and mechanical barriers against the invasion of fast-growing nonosteogenic cells (e.g., epithelial cells) and allow only the slow-migrating osteogenic cells to migrate into the defect sites. Commercially available GBR or GTR membranes are either resorbable e.g., poly-lactic acid (PLA), polylactic-glycolic acid (PLGA) or collagen or non-resorbable (e-PTFE or teflon).

Resorbable membranes can be divided into two groups: natural (collagen) or synthetic polymers (PLA, PLGA). Collagen has been reported to have the following properties: promote coagulation and wound healing, high tensile strength, chemotaxis for periodontal ligament cells, weak immunogenicity, and easy manipulation. (Garg, Biology, Harvesting, Grafting for Dental Implants, Rationale and Clinical Applications. Illinois, Quintessence Publications 2004; 59-63; Mellonig et al., Int J Perio & Restor Dent. 1993; 13:108-19). The fiber matrix of the polymers (natural or synthetics) is the primary structural component that provides adequate strength for space making during the initial phases of healing. While the synthetic polymers are broken down by hydrolysis, collagen is degraded by specific collagenolytic enzyme activity (Garg, Bone Biology, Harvesting, and Grafting for Dental Implants: Rationale and Clinical Applications. Illinois, Quintessence Publications 2004; 72-77).

Cell migration, attachment, and proliferation on biomaterials are important factors to achieve successful bone regeneration. The cell and tissue responses to GBR membranes have been studied demonstrating cell migration, proliferation and differentiation on GBR membranes (Takata et al., J Periodont Res 2001; 36: 322-327).

The osteoconductive calcium phosphate (CaP) biomaterials (hydroxyapatite, beta-tricalcium phosphate) allow attachment, proliferation, migration, and phenotypic expression of bone cells leading to formation of new bone in direct apposition to the CaP biomaterial (LeGeros, Clin Orthopaed Rel Res 2002; 395:81-98; Alliot-Licht et al., J Periodontol 1997; 68:158-165; Boyde et al., Bone 1999; 24: 579-589; Davies: The use of cell and tissue culture to investigate bone cell reactions to bioactive materials. In Yamamuro I, Hench L, Wilson J (eds); Handbook of Bioactive Ceramics. Boca Raton, CRC Press 1990; 1: 195-225; Hench et al., J Biomed Mater Res 1978; 2: 117-141; Osborn et al., Biomaterials 1980; 1:108-111; Price et al., J Biomed Mater Res. 1997; 37:394-400].

Guided bone regeneration failures commonly occur when nonresorbably and resorbable membranes are exposed to the oral environment. Exposure of guided bone regeneration membranes provide space that allows oral bacteria to attach, populate and cause further inflammation and bone resorption. Mombelli et al, J Periodontol. 1993: 64:1171-1175; Chen et al., J. Periodontol. 1997; 68:172-179. Hung et al., reported that oral bacteria such as Streptococcus mutans and Actionobacillus actinomycetemcomitans were able to penetrate through collagen or polymer membranes, and attachment of the PDL cell was affected on bacterial-contaminated GTR membranes. Hung et al., J Periodontol 2002; 73:843-851. Simon et al. observed bacterial penetration through polylactic-glycolic acid resorbable membranes and an increase in the amount of bacterial lining on the membrane from the first to the fourth week. Simon et al., Clin. Oral Implants Res. 1997; 8:23-31. Others reported that a greater loss of periodontal attachment was observed with greater levels of microorganism. Nowzari et al, J. Clin. Periodontol 1994; 21:203-210. Thus, controlling or eliminating priodontal pathogens on resorbable membranes is desirable.

Inhibition of the formation and metabolism of dental plaque and anti-bacterial effects by zinc salts has been well documented. Zinc salts have been included in several dental products because of their antibacterial properties. Afseth, Scand. J. Dent. Res. 1983; 91:169-174; Scand. J. Dent. Res. 1983; 91:42-45. Zinc ions released from zinc salt have shown to provide antibacterial property, inhibiting plaque formation and gingival inflammation. White, J. Clin. Dent. 1996; 7:27-31. Zinc coating on titanium alloy and orthodontic brackets were also shown to inhibit bacterial colonization. Alsilmi et al., J. Dent. Res. 2003; 82:2112.

It would be desirable to modify commercially available resorbable GBR membranes by mineralizing with a mineral such as calcium phosphate or zinc phosphate to enhance bone formation and reduce bacterial colonization.

### BRIEF SUMMARY OF THE INVENTION

The present invention features in a first aspect a method for mineralizing commercially available guided bone regeneration membranes. The method comprises the steps of (a) providing a commercially available guided bone regeneration membrane, (b) applying a mineralizing solution, and (c) exposing the membrane to microwave radiation. The method may further comprise (d) rinsing the membrane in a solution such as distilled water and (e) drying the membrane. The mineralizing solution may be a solution capable of supplying or delivering a mineral such as calcium or zinc. The microwaving step may be performed in any suitable microwave at any suitable power level and may be performed for any suitable duration, such as, for instance at least 5 minutes, at least 10 minutes, at least 12 minutes, at least 15 minutes, at least 20 minutes, etc. In some embodiments using a standard household microwave at medium power, 10-20 minutes, preferably 12-15 minutes is preferable. The mineralizing process may be performed until the mineral, such as, for instance calcium or zinc, is included in the membrane to a weight percent of at least 5%, at least 10%, at least 12%, at least 15%, at least 18%, at least 20% or at least 25% (weight percent) of the membrane. Similarly, the mineralizing process may be performed until the membrane reaches a width of at least about 1.25 times, at least 1.5 times, at least 1.67 times, at least 1.75 times or at least two times or even at least three times its original width as determined immediately before the mineralizing process was begun. The method of mineralizing as described herein comprising the step of exposing the membrane to microwave radiation is applicable to other medical and dental devices or materials including, but not limited to dental and orthopeadic implants, surface treatments, etc. and the method may be applied to modify existing bone grafting materials as one of ordinary skill in the art will readily understand.

In a second aspect, the invention provides a mineralized commercially available guided bone regeneration membrane. The mineralized guided bone regeneration membrane may be resorbable such as, for instance, poly-lactic acid (PLA), polylactic-glycolic acid (PLGA) or collagen or non-resorbable such as, for instance, e-PTFE or teflon. Similarly, the guided bone regeneration membrane may comprise natural polymers such as collagen or silk fibroin, or the guided bone regeneration membrane may comprise synthetic polymers such as, for instance, a polymer selected from the group consisting of PLA and PLGA, PFTE, and caprolactone. The mineralized guided bone regeneration membrane may be made by the method set forth, supra, comprising the steps of (a) providing a commercially available guided bone regeneration membrane, (b) applying a mineralizing solution, and (c) exposing the membrane to microwave radiation. The guided bone regeneration membrane may comprise a mineral, such as, for instance calcium or zinc at a weight percent of at least 5%, at least 10%, at least 12%, at least 15%, at least 18%, at least 20% or at least 25% (weight percent of the membrane). Naturally, more than one mineral may be present, including but not limited to calcium and/or zinc and/or phosphate and/or fluoride, and in the case where more than one mineral is present, the total weight percentages may equal those set forth, supra, or they may be additive to a total of, for instance, at least 5%, at least 10%, at least 12%, at least 15%, at least 18%, at least 20%, at least 25%, at least 30%, at least 40% or at least 50% (weight percent of the membrane). Additional ions or minerals, such as, for example, magnesium may be present in some embodiments. Similarly, the guided bone regeneration membrane, once mineralized, may have a width of at least about 1.25 times, at least 1.5 times, at least 1.67 times, at least 1.75 times or at least two times or even at least three times its original width as determined immediately before the mineralizing process was begun. The guided bone regeneration membrane may be biologically active to allow attachment, proliferation, migration and phenotypic expression of bone cells and be useful in leading to formation of new bone. Hence, the guided bone regeneration membrane may have applications in, for instance, dentistry and orthopedics. Also, in some embodiments, the guided bone regeneration membrane may be biologically active to promote increased collagen production. Similarly, in other embodiments, the guided bone regeneration membrane may be biologically active to inhibit bacterial colonization or bacterial infection. The guided bone regeneration membrane may be only one membrane layer thick, or it may be two, three, four or more membrane layers thick. That is, more than one individual membrane may be added to form the guided bone regeneration membrane according to the invention. In such embodiments where more than one membrane layer is provided, only one, two or even all individual membrane layers may be mineralized.

In a third aspect, the present invention provides a method for promoting or enhancing bone regeneration comprising providing a mineralized guided bone regeneration membrane. The guided bone regeneration membrane may comprise a mineral such as calcium or zinc. In preferred embodiments, the mineral, such as, for instance calcium or zinc or phosphate, is present in the membrane to a weight percent of at least 5%, at least 10%, at least 12%, at least 15%, at least 18%, at least 20% or at least 25% (weight percent) of the membrane. In some embodiments, additional minerals, such as, for instance, fluoride or magnesium may be present. Similarly, in some preferred embodiments, the membrane has a width of at least about 1.25 times, at least 1.5 times, at least 1.67 times, at least 1.75 times or at least two times or even at least three times its original width as determined immediately before the membrane was mineralized. In especially preferred embodiments, the membrane is one described, supra, in the second aspect of the invention, and it may be made according to the method set forth, supra, in the first aspect of the invention comprising the steps of (a) providing a commercially available guided bone regeneration membrane, (b) applying a mineralizing solution, and (c) exposing the membrane to microwave radiation. The method may be characterized by the presence of osteoblasts adhered to the membrane, and it may be characterized by increased production of collagen relative to methods using a guided bone regeneration membrane that has not been mineralized or that has not been mineralized by the method set forth, supra, according to the first aspect of the invention. In preferred embodiments, the bone regeneration is of a bone or bones in or near the oral cavity. However, in other embodiments any bone for which regeneration is sought may be the subject bone. In some embodiments, only one layer of membrane is used, however, in other embodiments multiple layers may be provided. In some embodiments, one of the multiple layers of membranes may comprise a membrane comprising one mineral according to the invention while another of the multiple layers may comprise a membrane having a second mineral according to the invention. In some embodiments, this aspect of the invention is accomplished by providing a mineralized guided bone regeneration membrane having one mineralized layer or one mineralized side. In such embodiments, the mineralized layer or the mineralized side is provided to face a bone defect.

In a fourth aspect, the present invention provides a method for inhibiting bacterial colonization or a method for inhibiting bacterial infection or a method for reducing inflammation comprising providing a mineralized guided bone regeneration membrane. The guided bone regeneration membrane may comprise a mineral such as calcium or zinc. In preferred embodiments, the mineral, such as, for instance calcium or zinc or phosphate, is present in the membrane to a weight percent of at least 5%, at least 10%, at least 12%, at least 15%, at least 18%, at least 20% or at least 25% (weight percent) of the membrane. In some embodiments, additional minerals, such as, for instance, fluoride or magnesium may be present. Similarly, in some preferred embodiments, the membrane has a width of at least about 1.25 times, at least 1.5 times, at least 1.67 times, at least 1.75 times or at least two times or even at least three times its original width as determined immediately before the membrane was mineralized. In especially preferred embodiments, the membrane is one described, supra, in the second aspect of the invention, and it may be made according to the method set forth, supra, in the first aspect of the invention comprising the steps of (a) providing a commercially available guided bone regeneration membrane, (b) applying a mineralizing solution, and (c) exposing the membrane to microwave radiation. The method may be characterized by the presence of osteoblasts adhered to the membrane, and it may be characterized by increased production of collagen relative to methods using a guided bone regeneration membrane that has not been mineralized or that has not been mineralized by the method set forth, supra, according to the first aspect of the invention. In preferred embodiments, the bone regeneration is of a bone or bones in or near the oral cavity. However, in other embodiments any bone for which regeneration is sought may be the subject bone. In some embodiments, only one layer of membrane is used, however, in other embodiments multiple layers may be provided. In some embodiments, one of the multiple layers of membranes may comprise a membrane comprising one mineral according to the invention while another of the multiple layers may comprise a membrane having a second mineral according to the invention.

### BRIEF DESCRIPTION OF THE DRAWINGS

FIG. 1 demonstrates a non-mineralized Resolut Adapt LT membrane (A), CaP mineralized membrane by using precipitation method (3 days) (B), 7 days (C), and microwave method (15 mins) (D).

FIG. 2 represents EDS analysis of CaP mineralized Resolut Adapt LT.

FIG. 3 represents XRD analysis of CaP mineralized Resolut Adapt LT.

FIG. 4 represents FT-IR of CaP mineralized Resolut Adapt LT showing absorption bands of functional groups belonging to the polymer and carbonate apatite.

FIG. 5 depicts a non-mineralized Biomend Extend membrane (A), CaP mineralized membrane by using precipitation method (3 days) (B), 7 days (C), and microwave method (15 mins) (D).

FIG. 6 represents EDS analysis of CaP mineralized Biomend Extend.

FIG. 7 represents FT-IR of CaP mineralized Biomend Extend showing absorption bands of functional groups belonging to the polymer and carbonate apatite.

FIG. 8 represents XRD analysis of CaP mineralized Biomend Extend.

FIG. 9 represents CD1 gene expression on CaP mineralized and non-mineralized membranes (STD about 20% of values), n=10 for each group; No significant difference p>0.05.

FIG. 10 represents CDK4 gene expression on CaP mineralized and non-mineralized membranes (STD about 20% of values), n=10 for each group; No significant difference p>0.05.

FIG. 11 represents collagen type I expression on mineralized and non-mineralized membranes.

FIG. 12 represents light microscopic (A, original magnification X10 and B, original magnification X40) image showing cell attachment on the collagen membranes (Biomend Extend).

FIG. 13 represents light microscopic (A, original magnification X10 and B, original magnification X40) image showing cell attachment on the co-polymer membranes (Resolut Adapt LT).

FIGS. 14 A, B, and C are SEM images showing cell attachment on mineralized co-polymer membranes.

FIGS. 15 A, B are SEM images showing cell attachment on mineralized collagen membranes.

FIG. 16 is scanning electron microscopy images of nonmineralized copolymer membrane, Resolute Adapt L T (A), zinc phosphate mineralized copolymer membrane using the precipitation method, 3 days (B), 7 days (C), and the microwave method for 15 minutes (0).

FIG. 17 represents energy dispersive system spectrum of zinc phosphate mineralized Resolute Adapt L T (copolymer) showing the presence of zinc and phosphate elements.

FIG. 18 is scanning electron microscopy images of nonmineralized collagen membrane, BioMend Extend (A), zinc phosphate mineralized collagen membrane using the precipitation method, 3 days (B), 7 days (C), and the microwave method for 15 minutes (D).

FIG. 19 is an x-ray diffraction profile of zinc phosphate mineralized membrane (A). Fourier transform infrared spectrum of zinc phosphate mineralized membranes (8).

FIG. 20 depicts colony forming units of the bacterial colonies on nonmineralized (BC) and mineralized (BT) collagen membrane, BioMend Extent, as a function of incubation period.

FIG. 21 is an interactive graph of mean bacterial level as a function of incubation period on mineralized (test) and nonmineralized (control) membranes (error bars show mean+/−2.0 standard deviation). *Test=ZnP mineralized collagen membranes; *Control=non-mineralized collagen membranes.

FIG. 22 depicts colony forming units of the bacterial colonies on nonmineralized (RC) and mineralized (RT) copolymer membranes, Resolute Adapt L T, as a function of incubation period.

FIG. 23 is an interactive graph of mean bacterial level as a function of incubation period on mineralized (test) and nonmineralized (control) copolymer membranes, Resolute Adapt L T (error bars show mean+/−2.0 standard deviation).

FIG. 24 demonstrates that the membranes are biologically active to allow attachment, proliferation, migration or phenotypic expression of bone cells. The data depict the effect of membranes mineralized with calcium phosphate on (a) the expression of human osteoblast proliferation markers CD1 and CDK4 (both are growth markers) (C=control, ABC=absolute control (mineralized silk fibroin), AB/CAP=absolute mineralized with CAP, and AB/CAP+NAF=absolute control with CAP and with fluoride); (b) the expression of human osteoblast phenotype markers Col I (type I collagen) and AP (alkaline phosphatase) (C=control, ABC=absolute control (mineralized silk fibroin), AB/CAP=absolute mineralized with CAP, and AB/CAP+NAF=absolute control with CAP and with fluoride); and (c) human osteoblast proteoglycan markers lumican, decorin, biglycan, and versican (C=control, ABC=absolute control (mineralized silk fibroin), AB/CAP=absolute mineralized with CAP, and AB/CAP+NAF=absolute control with CAP and with fluoride).

FIG. 25 demonstrates that the membranes are biologically active to stimulate or enhance Type I collagen production. The data depicts the effect of membranes mineralized with calcium phosphate on collagen production (measured in ng/ml) by human osteoblasts (C=control, ABC=absolute control (mineralized silk fibroin), AB/CAP=absolute mineralized with CAP, and AB/CAP+NAF=absolute control with CAP and with fluoride).

### DETAILED DESCRIPTION

The present provides modified resorbable commercial co-polymers (Resolut Adapt LT) and collagen (Biomend Extend) membranes having bioactive properties and methods of making the same. A bioactive property was introduced by mineralizing the membranes with calcium phosphate, specifically carbonate apatite as identified by FT-IR. Carbonate apatite is the main mineral in bone. Carbonate apatite as bone graft materials has been shown to enhance bone formation (LeGeros, Calcium Phosphate in Oral Biology and Medicine. Monographs in Oral Biology Vol 15. H. Myers (ed). 1991; Karger: Basel; Sakae et al., Key Engineer Mat 2003; 240-242: 395-398).

As used herein “guided bone regeneration membranes” include all varieties available for use in surgical procedures. Guided bone regeneration membranes may be derived from a variety of sources, both natural and synthetic, and are marketed under various trade names. Membranes used in guided bone regeneration and grafting may be of two principal varieties, non-resorbable and resorbable. Non-resorbable membranes include expanded polytetrafluoroethylene (ePTFE) membranes which are biocompatible synthetic polymer membranes of the Teflon trade name. Resorbable or bioabsorbable membranes were developed to avoid the limitations of ePTFE which must usually be surgically retrieved in 4-6 weeks. Resorbable membranes are normally either animal-derived or synthetic polymers. They are gradually hydrolyzed or enzymatically degraded and therefore do not require a second surgical removal. There are many sources of such membranes including rat collagen, bovine collagen, ox cecum cargile membrane, polylactic acid, polyacetic acid, polyglycolic acid, polyglactin 910(Vicryl), synthetic skin (Biobrane) and freeze-dried dura mater. Recently developed synthetic membranes are often combinations of various materials. Collagen resorbable membranes are normally of either type I or II collagen from bovine or porcine sources. They are often cross-linked and resorbed between 4-38 weeks. Brands of collagen barriers include Biomend, Biomend Extend, OSSIX, Neomem, and Hypro-Sorb. Synthetic resorbable membranes may be polymers of lactic acid or glycolic acid. Their ester bonds are often degraded over 30-60 days, leaving free acids that may be pro-inflammatory. There are several types of synthetic membranes including Vicryl, Atrisorb, Atrisorb-FreeFlow, Arisorb-D, Resolut XT, Epi-Gide and Gore Resolut Adapt, each made predominantly of acid polymers. In addition, Capset is a calcium sulphate derivative synthetic membrane.

Different methods of mineralization that have been used including, for instance, painting, precipitation, and mechanical mixing (e.g., mechanical mixing calcium phosphate powder in collagen gel). The present invention provides a new method of mineralization using a microwave. A microwave method has been used for synthesis of apatite and conversion of coral to calcium phosphate (Eppley et al., J Craniofacial Surg 2002; 13:5:681-686; Siddharthan et al., J Mat Sci Mat Med 2004; 1279-1284; Pena et al., Key Engineer Mat 2001; 192-195: 347-350).

Calcium Phosphate Mineralized Guided Bone Regeneration Membranes

The present invention demonstrates that the microwave method compared to the precipitation method is more efficient for mineralizing resorbable commercial membranes in terms of higher mineral contents obtained and shorter time required. For example, the average amount of CaP mineral incorporated with a collagen membrane (Biomend Extend) was 13.99±1.23 wt % using microwave (15 mins), 7.5±0.5 wt % using a precipitation method (7 days). Similar differences are observed with mineralization of a co-polymer membrane (Resolut Adapt LT) as follows: a microwave method (17.23±2.56 wt %) compared to precipitation method (11.45±1.26 wt %). Mineralization time is critical for resorbable membranes, especially for collagen, because membranes in solution begin a degradation process with time resulting in changing physical and chemical properties. Therefore, the much shorter time required for mineralization using the microwave method is a decided advantage. Mineralization of collagen membranes using either method results in an increased in stiffness. However, these physical changes do not appear to affect the ability of the membrane to serve as barrier as demonstrated by histological analysis.

The microwave method can also be used to mineralized all types of polymers (natural and synthetics) with different mineral composition by adjusting the composition, pH, and temperature of the calcifying solution. Takata et al. have observed periodontal ligament cell attachment, proliferation and differentiation on various commercial membranes including collagen (Biomend), and co-polymer membrane (Resolut). They also reported migration of osteoblastic cells on those membranes (Takata et al., J Periodont Res 2001; 36: 322-327; Takata et al., Clin. Oral Impl. Res. 2001; 12: 332-338).

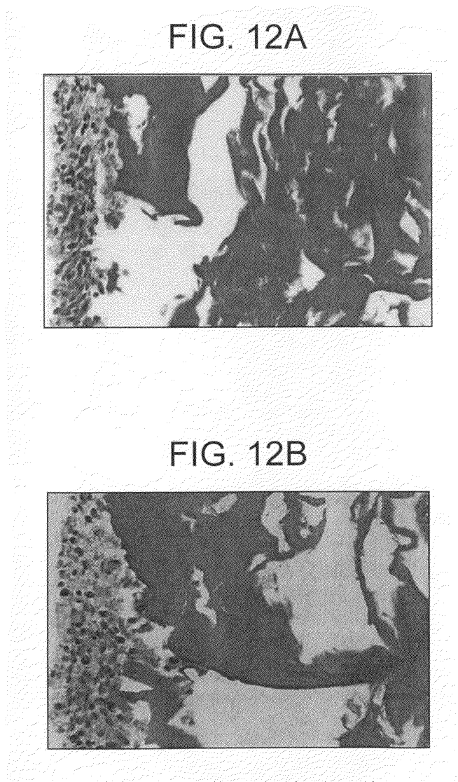

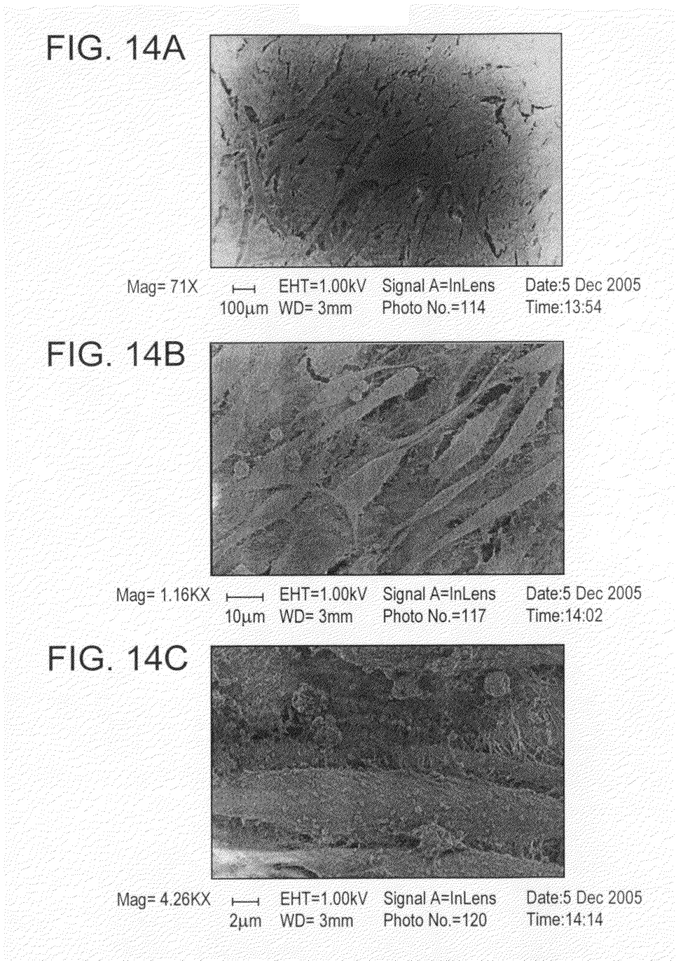

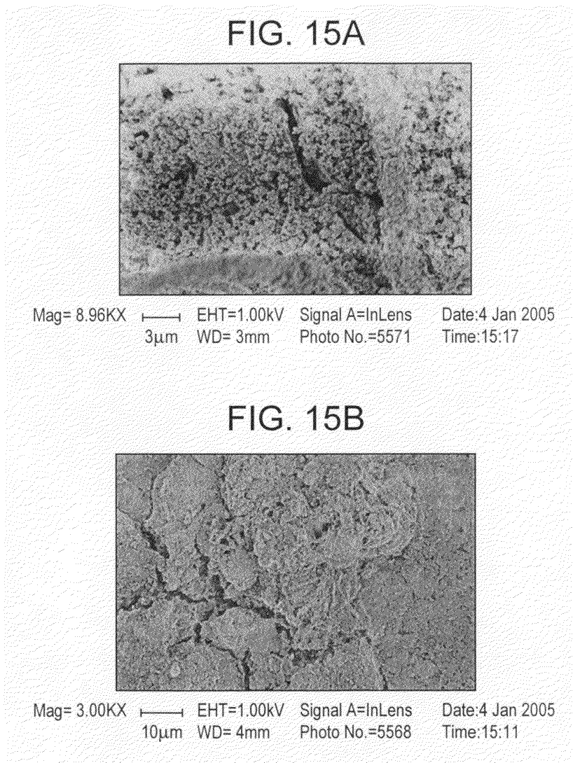

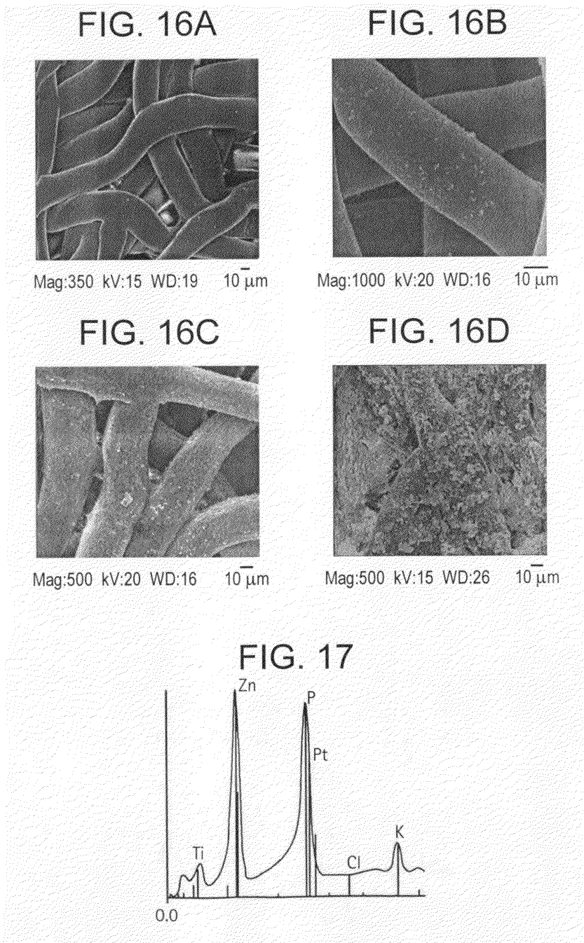

Urist et al. described osteoconductivity as the property of material to support tissue ingrowth, osteoprogenitor cell growth and development for bone formation to occur (Urist et al., Science 1965; 150:893-898). The osteoconductive CaP biomaterials allow attachment, proliferation, migration, and phenotypic expression of bone cells leading to formation of new bone in direct apposition to the CaP biomaterial. The advantage of mineralizing these membranes with carbonate apatite was demonstrated in the results of an in vitro cell response study. Histologic examination and SEM analyses showed greater cell attachments on both mineralized co-polymer and mineralized collagen membranes compared to non-mineralized membranes (FIGS. 12-15). The morphology of attached cells on mineralized co-polymer and mineralized collagen membranes was different (FIGS. 14, 15). The cells had flat, stellate, fibrous shape on mineralized co-polymer membranes compared to condensed round shape on mineralized collagen membranes. Takata et al, supra, also found similar results that the morphology of cells varied on different types (synthetic vs. natural polymers) of GBR membranes. The differences in surface characteristics and composition of the two types of membranes may cause the observed differences in cell morphology. In addition, the mineralized membranes showed statistically significant increase in type I collagen expression compared to the non-mineralized membranes (FIG. 11). The present invention demonstrates the advantage of carbonated apatite as a bioactive material for promoting cell attachment and differentiation and in promoting bone regeneration.

The present invention demonstrates that CaP mineralized resorbable commercial membranes (collagen or co-polymer) elicit favorable osteoblast cell response and increased type I collagen expression compared to the non-mineralized membranes. These results indicate that the CaP mineralized membranes promote and enhance bone regeneration in vivo.

The present invention demonstrates that CaP mineralized resorbable commercial membranes (collagen or co-polymer) elicit favorable osteoblast cell response and increase type I collagen expression compared to the non-mineralized membranes. These results indicate that the CaP mineralized membranes promote and enhance bone regeneration in vivo. The present invention also demonstrates the microwave method of mineralization is more efficient than the precipitation method.

Zinc Phosphate Mineralized Guided Bone Regeneration Membranes

Zinc salts have been included in several dental products because of their antibacterial properties. (Afseth, Scand. J. Dent. Res. 1983; 91:169-174) Zinc coating on titanium alloy and orthodontic brackets have also been shown to inhibit bacterial colonization. (Alsilmi et al., J. Dent. Res. 2003; 82:2112).

The combined energy dispersive system, x-ray diffraction, and Fourier transform infrared analyses identified zinc phosphate as the mineral in membranes mineralized in solutions containing zinc and phosphate ions. Inductive coupled plasma analysis showed the amount of ions released from mineralized membranes in solution at different time periods (days 3, 7, 10, 13, and 15). Release of zinc and phosphate ions from the mineralized membranes were observed even after day 15, but the maximum release of these ions occurred on day 3.

A. actinomycetemcomitans is the most dominant bacterial strain found in failed or exposed guided tissue, or guided bone regeneration resorbable or nonresorbable membranes. (Sela et al., Clin. Oral Implants Res. 1999; 10:445-452) For this reason, A. actinomycetemcomitans, a standard strain ATCC 29522, was used to determine the antibacterial property of membrane mineralized with zinc phosphate. A. actinomycetemcomitans is an anaerobic gram-negative bacterium that is a strong marker of periodontitis in adults and is linked to progression of the periodontal disease. Bacterial accumulation and invasion of the exposed guided bone regeneration membrane can limit the regenerative process in both periodontal and implant surgical treatment. (Sela, supra)

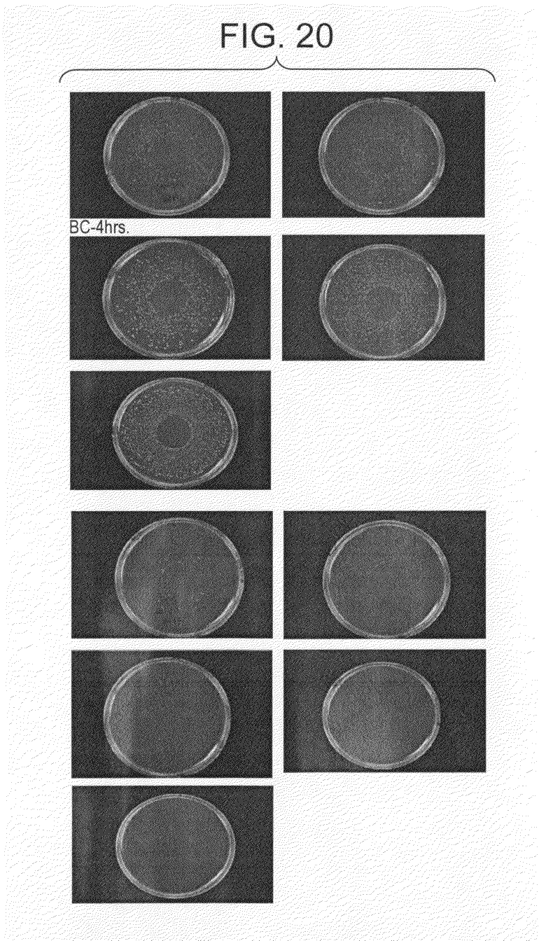

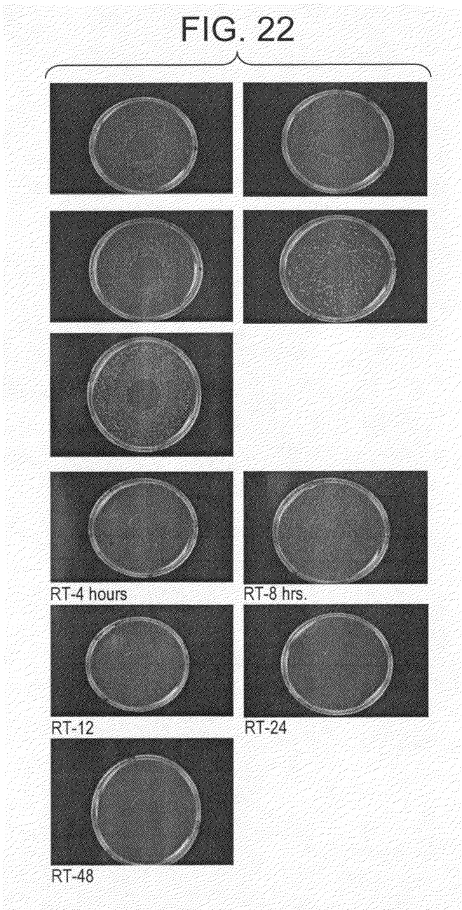

The present invention demonstrates the antibacterial effect of zinc phosphate mineralized collagen (BioMend Extend) and zinc phosphate mineralized copolymer membranes (Resolut Adapt LT). The membranes with zinc phosphate show statistically significant inhibition of bacterial colonization compared to non-mineralized membranes. In non-mineralized membranes, the number of colony forming units of the bacteria increase with increasing incubation times (FIGS. 20 and 22). The zinc phosphate mineralized collagen membrane (BT) show a significant antibacterial effect in 24 hours, while the zinc phosphate mineralized copolymer membrane (RT) showed a significant antibacterial affect after 48 hours. BT and RT membranes showed a statistically significant difference at P>0.001. Zinc phosphate incorporated with collagen membranes appear to have a better antibacterial effect.

The present invention demonstrates that the use of zinc phosphate mineralized membranes (collagen or copolymers) can inhibit bacterial colonization that can lead to premature degradation of the membranes, inflammation, and inhibition of soft and hard tissue regeneration. In addition, zinc has been shown to promote healing, and therefore, zinc phosphate mineralized membranes provide an antibacterial effect and accelerated healing. (Valee et al., Physiol. Rev. 111993: 73:79-118).

The present invention further demonstrates that zinc phosphate mineralized membranes have a significantly greater antibacterial effect than nonmineralized membranes. This indicates that zinc phosphate mineralized membranes inhibit bacterial colonization in vivo, thus preventing inflammation and bone resorption.

The invention may be better understood by reference to the following examples, which are intended to be exemplary of the invention and not limiting thereof.

### EXAMPLES

The following examples are intended to provide those of ordinary skill in the art with a complete disclosure and description of how to make and use the therapeutic methods of the invention and membranes, compounds and pharmaceutical compositions, and are not intended to limit the scope of the invention. Efforts have been made to ensure accuracy regarding numbers used (e.g., amounts, temperature, etc.), but some experimental errors and deviations may occur. Unless indicated otherwise, parts are parts by weight, molecular weight is average molecular weight, temperature is in degrees Centigrade, and pressure is at or near atmospheric.

### Example 1

### Calcium Phosphate Mineralized Guided Bone Regeneration Membranes

### Materials and Methods

Commercial membranes, Resolut Adapt LT (W.L. Gore, Flagstaff, Ariz., USA) consisting of co-polymers polyglycolic acid/trimethylene carbonate(PGA/TMC) and Biomend Extend (Zimmer, Carlsbad, Calif., USA) consisting of bovine-derived collagen, were used. The membranes were cut into sections, 1cm², and mineralized using the precipitation and microwave method described below. The mineralized membranes were rinsed in double distilled water and air dried. The mineralized membranes were characterized using Scanning Electron Microscopy, SEM (JEOL-JSM-5400, USA and Hitachi S-3500N, Japan), Energy Dispersive System, EDS (PGT Prism, N.J., USA), Fourier Transform Infra-Red (FT-IR) Spectroscopy (Nicolet 550, France), Thermogravimetry, TGA, (SQ 600, TA Instrument, Texas, USA), and Inductive Coupled Plasma, ICP (Thermo Jarrell ash Model-Trace Scan Inductive Coupled Plasma, Waltham, Mass., USA).

(i) Precipitation Method

Each membrane section (1 cm²) was incubated in 20 ml calcifying solution for 3 days and 7 days at 37° C. The calcifying solution consisted of calcium acetate (4 mM), sodium phosphate (4 mM), and sodium bicarbonate (4 mM). All chemical reagents were purchased from Fisher Scientific, NJ, USA. At the end of 3 days and 7 days, the calcium phosphate (CaP) mineralized membranes were rinsed in double distilled water and air dried.

(ii) Microwave Method

The microwave method of mineralization described, supra, was used. Each membrane section (1 cm²) was immersed in 20 ml calcifying solution contained in a beaker and placed in the microwave oven (Carousel, Sharp, Japan), operated at medium power until the solution has completely evaporated. The CaP mineralized membranes were rinsed in double distilled water and air dried.

### Determination of In Vitro Cell Culture Response to CaP Mineralized Membranes Cell Culture:

A human osteoblast surrogate cell line (MG-63) was cultured and propagated in Dulbecco's Modified Eagle's Medium: Nutrient Mix F-12 (DMEM/F-12; Invitrogen/Gibco, San Diego, Calif., USA) containing 15 mM HEPES, L-glutamine, and pyridoxine hydrochloride. The media was supplemented with 10% fetal calf serum (FCS; Invitrogen/Gibco, San Diego, Calif., USA), sodium bicarbonate (1.5 g/l; Sigma-Aldrich, St. Louis, Mo., USA), and sodium pyruvate (1.0 mM; Sigma-Aldrich, St. Louis, Mo., USA). At confluence, cells were harvested by trypsinization and plated onto the test scaffold material or onto control Thermanox disks in 24-well polystyrene plates at a density of 5×10⁵ cells in 50 μl. The cell suspension was allowed to percolate through the membrane and allowed to stand for another 10 min before adding 1 ml of media. Cells were incubated at 37° C., 5% CO₂ for 14 days. The cultures were replenished every 4 days by adding 100 μl of fresh media. At the end of the experiment, cells seeded disks or test scaffold materials were recovered for: (1) histological, (2) SEM and (3) gene expression analyses. The supernatant was frozen for subsequent type I collagen determination using enzyme linked immune assay (ELISA).

### Gene Expression Analyses (RNA Analysis)

RNA was isolated using the TRIzol© reagent method (Invitrogen/Life Technologies™, Rockville, Md., USA) where 1 ml of Trizol was added for every 0.5×10⁶ cells seeded disk or scaffold ensuring all the cells were completely lysed. Isolated RNA was converted to single-stranded cDNA using the Advantage RT-for-PCR Kit (Clontech Laboratories, Palo Alto, Calif., USA). Specific primers for the housekeeping gene GAPDH, osteoblast phenotypic markers (type I collagen (Col I).

### Results

### Calcium Phosphate (CaP) Mineralized Resolut Adapt LT (Polyglycolic Acid/Trimethylene Carbonate Co-Polymer)

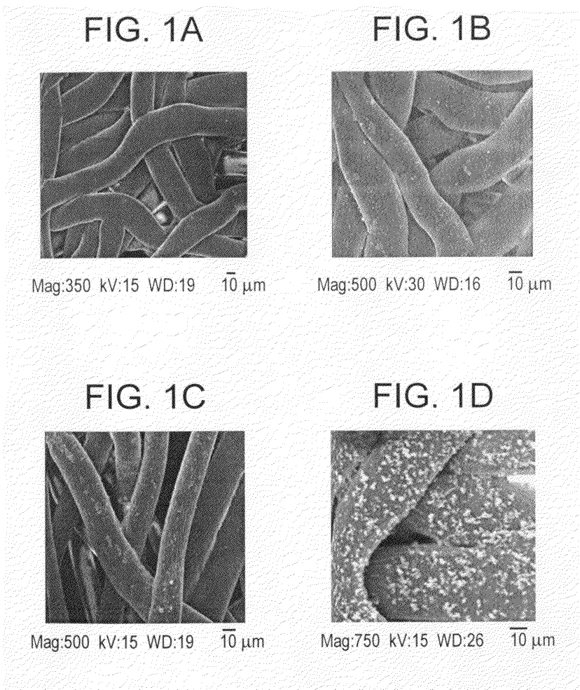

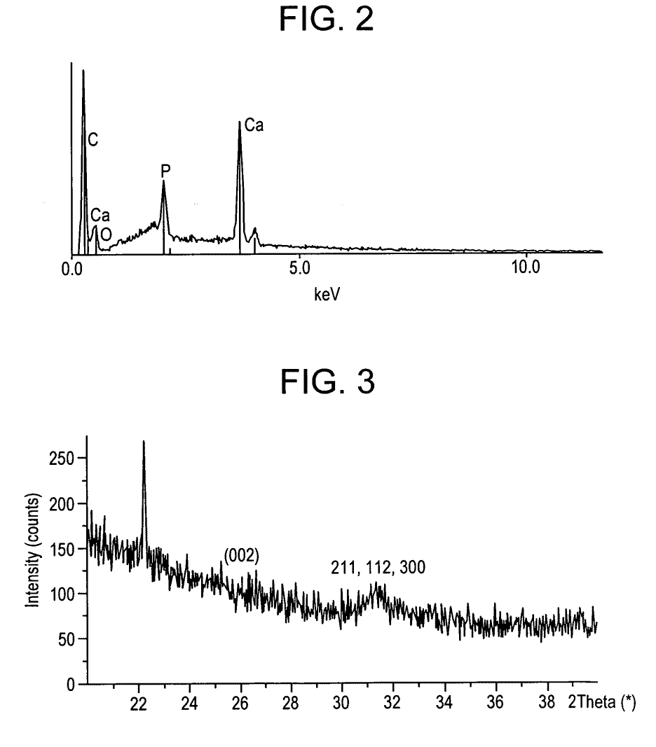

SEM of commercial membrane (Resolut Adapt LT) before and after mineralization by precipitation and microwave methods are shown in FIG. 1. The size and quantity of crystals formed on the membrane surfaces were greater using the microwave method for 12 to 15 minutes compared to that by precipitation method even after seven days (FIGS. 1B, 1C compared to FIG. 1-D). The membranes mineralized with either method showed thin calcified films. The crystals and the films were identified by EDS to consist mainly of carbon, calcium and phosphate ions (FIG. 3). The relative calcium to phosphate ratio of the peaks intensities of the spectra are similar to that obtained for calcium hydroxyapatite or carbonated apatite. The amount of crystals formed after seven days in calcifying solutions was greater than those obtained after 3 days. The adherence of the mineral CaP to the membranes was maintained even after vigorous rinsing with double distilled water. The membranes maintained their original texture and morphology after mineralization by either method (FIG. 1).

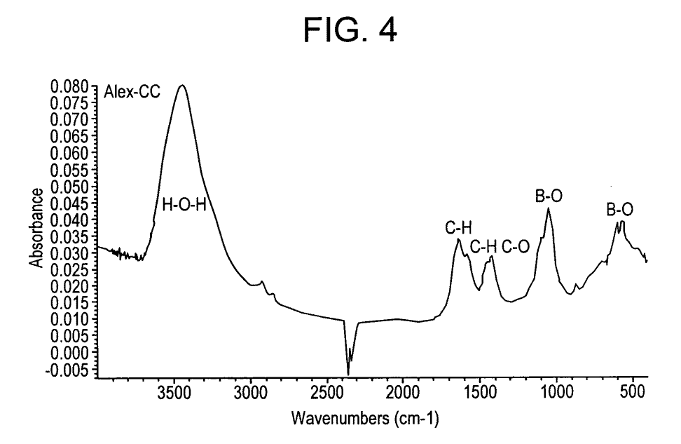

XRD analysis showed the presence of apatitic calcium phosphate (apatite with low crystallinity indicating small crystal size) shown in FIG. 4. The high background is due to the presence a large amount of polymer.

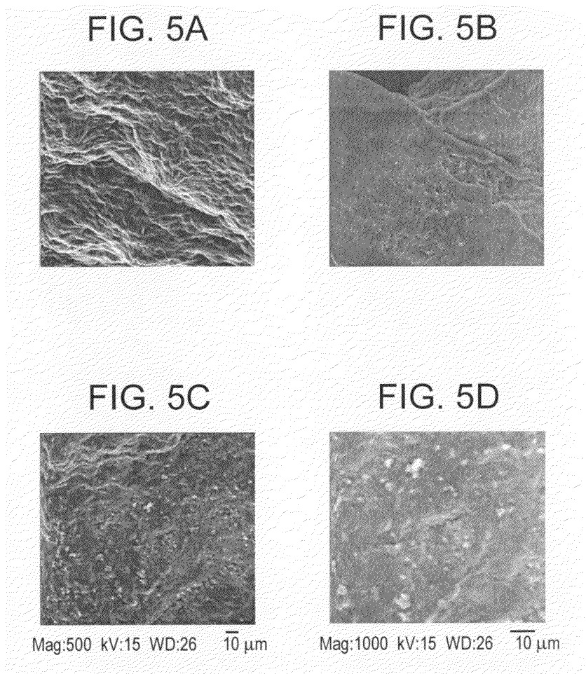

FT-IR spectra of mineralized membranes showed absorption bands of the polymer (1500 to 1800 cm⁻¹), phosphate (1000 to 1200 cm⁻¹ and 500 to 600 cm⁻¹,), carbonate (1400 to 1500 cm⁻¹ and at 860 to 880 cm⁻¹), and adsorbed water (at 3200 to 3600 cm⁻¹) shown in FIG. 5.

TGA analysis showed that the mineralization by microwave method (12-15 mins) allowed the inclusion of significantly (p<0.005) more carbonate apatite (average weight, 17.23±2.56 wt %) compared to the precipitation method even after 7 days (average weight 11.45±1.26 wt %).

### Calcium Phosphate (CaP) Mineralized Biomend Extend (Bovine Collagen)

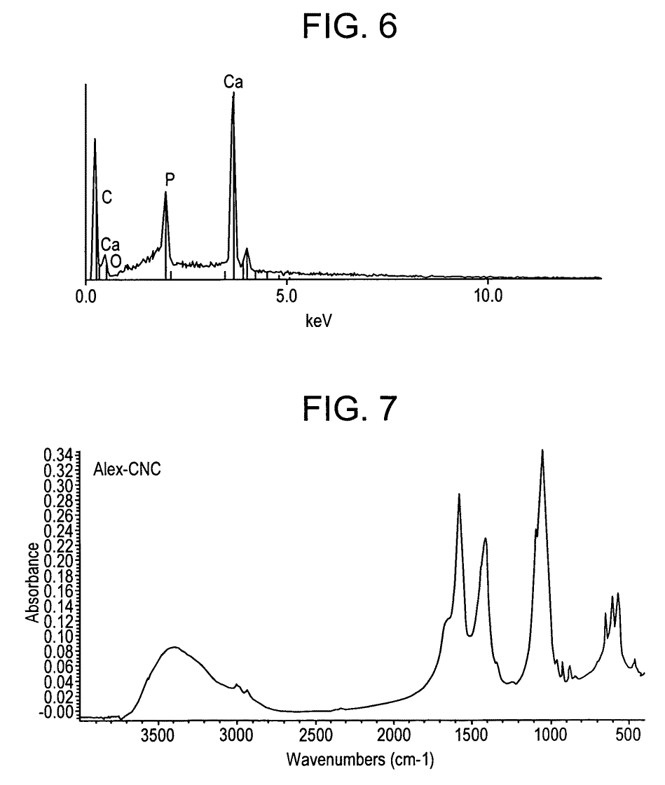

SEM analysis showed that the surface morphology of the collagen membrane was changed after mineralization using either with precipitation or microwave methods (FIGS. 5B, 5C compared to 5D). The width of the membrane increased to twice its original size and the texture of the membrane became rigid and solid to touch. Calcified films and crystals were obtained after mineralization by either methods (FIGS. 5B, 5C, and 5D). The amount of crystals on the membranes increased with increasing precipitation time (3 days vs 7 days) as shown in FIGS. 5B and 5C. The crystals formed using microwave method for 15 minutes (FIG. 5D) were larger than those formed after seven days precipitation (FIG. 5C). The amount of crystals formed after seven days precipitation were greater than those obtained after 3 days. Separate EDS analysis of the calcified film and crystals showed the presence of mainly carbon, calcium and phosphorus in the EDS spectrum (FIG. 6). The relative calcium to phosphorus ratio of the peaks is similar to that obtained for calcium hydroxyapatite or carbonated apatite.

FT-IR spectra of mineralized membranes showed absorption bands of the collagen (at 1500 to 1800 cm⁻¹), phosphate (at 1000 to 1200 cm⁻¹ and at 500 to 600 cm⁻¹), carbonate (at 1400 to 1500 cm⁻¹ and at 860 to 880 cm⁻¹), and adsorbed water (at 3200 to 3600 cm⁻¹) shown in FIG. 7.

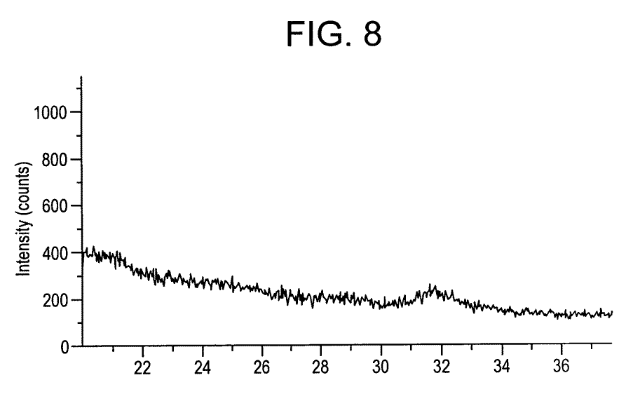

XRD analysis showed the presence of apatitic calcium phosphate (apatite with low crystallinity indicating small crystal size (FIG. 8). The high background is due to the presence a large amount of collagen.

TGA analysis showed that the mineralization by microwave method, 12-15 mins (13.99±1.23 wt %) allowed the inclusion of significantly (P<0.001) more carbonate apatite compared to the precipitation method even after 7 days (7.57±0.5 wt %).

Release of Ions from Mineralized Membranes

ICP analysis of solution after incubation of CaP mineralized collagen membrane (Biomend Extend) and CaP mineralized co-polymer membranes (Resolut Adapt LT) showed release of calcium and phosphate ions up to 15 days (Tables 1A, 1B). The maximum release of calcium ions was observed in day 3 solution for both mineralized membranes: CaP mineralized collagen, 4.23±0.07 ppm; and co-polymer membranes, 5.43±0.02 ppm. The maximum release of phosphate ions was also observed in day 3 solution for both mineralized membranes: mineralized collagen membranes, 1.71±0.02 ppm; and mineralized co-polymer membranes, 1.54±0.02 ppm. The amount of ions released decreased with time.

TABLE 1A
Calcium and phosphate ions released (in ppm) in solution
from mineralized co-polymer membranes (±std)

| | Day 3 | Day 7 | Day 10 | Day 13 | Day 15 |
|---|---|---|---|---|---|
| Ca | 4.23 ± 0.07 | 1.43 ± 0.02 | 1.96 ± 0.01 | 0.59 ± 0.01 | 0.34 ± 0.01 |
| P | 1.71 ± 0.02 | 1.18 ± 0.01 | 0.60 ± 0.01 | 0.43 ± 0.01 | 0.23 ± 0.01 |

TABLE 1B
Calcium and phosphate ions released (in ppm) in solution
from mineralized collagen membranes (±std)

| | Day 3 | Day 7 | Day 10 | Day 13 | Day 15 |
|---|---|---|---|---|---|
| Ca | 5.43 ± 0.02 | 1.89 ± 0.05 | 0.54 ± 0.02 | 0.50 ± 0.01 | 0.16 ± 0.01 |
| P | 1.54 ± 0.02 | 1.09 ± 0.01 | 0.67 ± 0.01 | 0.77 ± 0.05 | 0.52 ± 0.01 |

In Vitro Cell Response

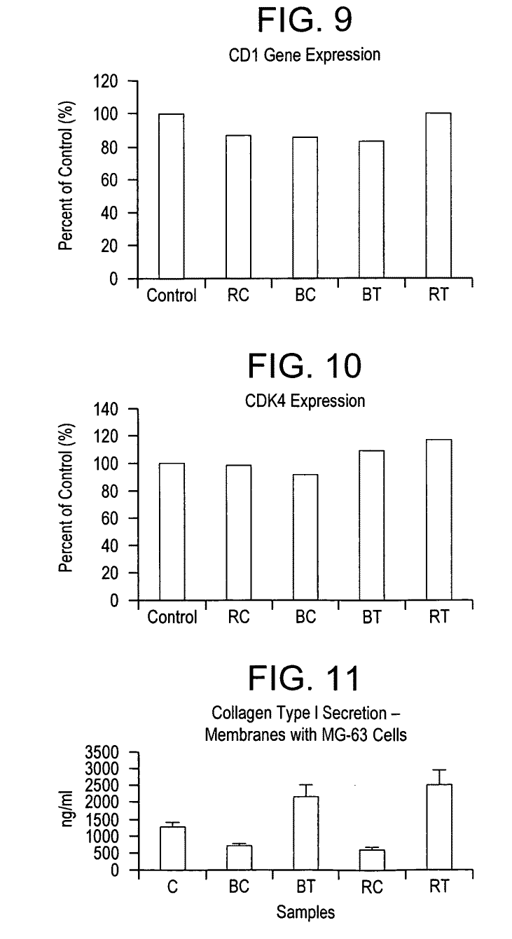

Cell proliferation was indicated by CDK4, and CD1 gene expression (FIGS. 9, 10). No significant difference (P>0.05) was observed with CD1 and CDK4 gene expression between mineralized (BT) and non-mineralized (BC) collagen membranes (FIGS. 9, 10). Collagen type I expression was higher for mineralized membranes (BT and RT) compared to non-mineralized membranes (BC and RC) (p<0.05) as shown in FIG. 11. However, there was no significant difference between RT and BT in type I collagen expression.

### Histology

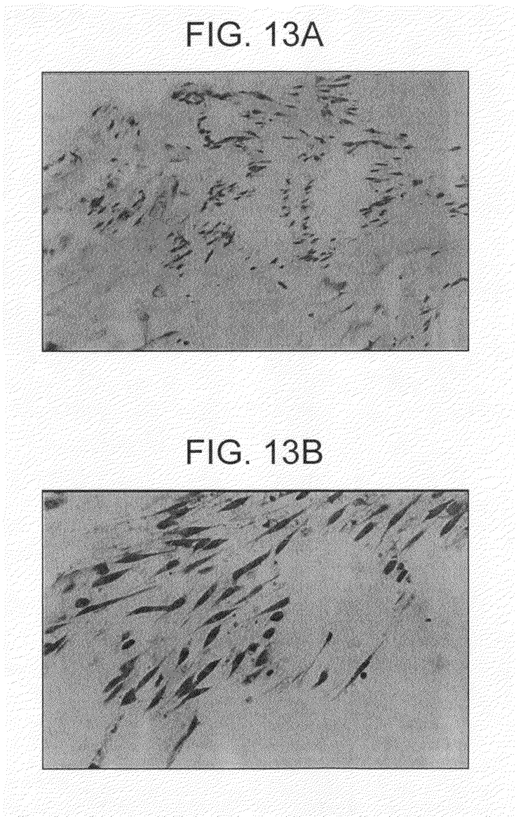

Histologic analysis demonstrated that cells on collagen membranes (Biomend Extend), were densely attached on membrane surfaces (FIGS. 12A, B). Unlike collagen membranes, cells on co-polymer membranes (Resolut Adapt LT) were randomly attached and cells were also observed in the spaces between the polymer fibers (FIGS. 13A, B).

### SEM Examination

The morphology of attached cells on co-polymer and collagen membranes was different. The cells on the mineralized co-polymer membranes appeared flat and elongated in shape (FIGS. 14 A, B, and C) compared to the cell on mineralized collagen membranes which appeared round in shape (FIGS. 15 A, B). These differences in the cell shapes suggest that the membrane surface morphology will have an effect on cell spreading.

### Example 2

### Preparation of Zinc Phosphate Mineralized Membranes

### Materials and Methods

Commercial membranes, Resolut Adapt L T (W.L. Gore & Associates, Inc., Flagstaff, Ariz.), consisting of copolymers (PGAffMC), and BioMend Extend (Zimmer Dental, Carlsbad, Calif.), consisting of bovine-derived collagen, were used. The membranes were cut into sections, 1 cm², and mineralized using the precipitation and microwave method described below. The mineralized membranes were rinsed in double distilled water and air dried. The mineralized membranes were characterized using scanning electron microscopy (JOEL JSM-5400; JOEL USA, Inc., Peabody, Mass.; and Hitachi S-3500N; Hitachi, Ltd., Tokyo, Japan), energy dispersive system (Princeton Gamma-Tech Instruments, Inc. [PGT], Princeton, N.J.), Fourier transform infrared spectroscopy (Nicolet 550; France), thermogravimetry (SQ 600′; Texas Instruments, Inc., Dallas, Tex.), and inductive coupled plasma (Thermo Jarrell Ash Model-Trace Scan Inductive Coupled Plasma, Waltham, Mass.).

Precipitation method. Each membrane section (1 cm²) was incubated in 20 mL zinc phosphate solution for 3 and 7 days at 37° C. The zinc phosphate solution consisted of zinc acetate and potassium phosphate. At the end of 3 and 7 days, the zinc phosphate mineralized membranes were rinsed in double-distilled water and air dried.

Microwave method. The microwave method for mineralizing membranes described, supra, was used. Each membrane section (1 cm²) was immersed in 20 mL zinc phosphate solution contained in a beaker and placed in the microwave oven (Carousel; Sharp), operated at medium power until the solution completely evaporated. The zinc phosphate mineralized membranes were rinsed in double-distilled water and air dried.

Thermogravimetric analysis was used to determine the total weight percent of zinc phosphate in the mineralized membranes. Five specimens of zinc phosphate mineralized membranes were used for analysis. The thermogravimetry was set to ramp 20° C./minutes up to 600° C. and isothermal for 30 minutes. The weight changes were recorded and the data analyzed to calculate total mineral contents.

### Inductive Coupled Plasma Analysis

To determine the release of zinc and phosphate ions from the zinc phosphate mineralized membranes, zinc phosphate mineralized and nonmineralized (control) membranes were incubated in separate test tubes containing 20 mL double-distilled water. Five membrane sections from each group were used in the analysis. Every day for 15 days, the membrane sections were transferred into new test tubes containing the same volume of double-distilled water. The amounts of zinc and phosphate ions released from zinc phosphate mineralized membranes were determined using inductive coupled plasma.

### Microbial Response of Zinc Phosphate Mineralized Membranes

Actinobacillus actinomycetemcomitans A TCC 29522 standard strain (American Type of Culture Collection, Manassas, Va.) was purchased. The bacteria were suspended in 100 mL phosphate-buffered saline buffer and inoculated into tryptic soy-serum bacitracin vancomycin agar (Anaerobe Systems, Concord Circle Morgan Hill, Calif.). Gram stain identified A. actinomycetemcomitans after incubation at 37° C. for 72 hours in an anaerobic atmosphere of 85% N₂, 10% Hz, and 5% CO₂, and then cultured in brain heart infusion broth (Anaerobe Systems) overnight under the same anaerobic condition with different durations. The concentration of the bacterial culture was adjusted to 0.35 at OD₆₆₀. Two sets of 10-fold serial dilutions were prepared, and 4.4-mL aliquots of the bacterial culture were ready to use. Each 8 mineralized membranes and 8 nonmineralized membranes (control) were put into the test tubes at the same time in the anaerobic chamber for cultivation. The membranes were taken out from the test tubes at 4, 8, 12, 24, and 48 hours. Each membrane was washed by pipetting 4 mL phosphate-buffered saline buffer 3 times to remove the nonadherent bacterial cells, transferred into a new test tube, which contained 500 mL brain heart infusion broth, and sonicated (Sonicator ultrasonic processor; Misonix Inc., Farmingdale, N.Y.) for 30 seconds to remove attached bacteria on the membranes. Ten-fold serial dilutions of the bacterial samples were prepared and plated on the tryptic soy-serum bacitracin vancomycin medium using the Spiral Autoplate 4000 (Spiral Biotech, Inc., Bethesda, Md.). The plates were incubated in the same anaerobic condition for 72 hours. The colony forming units of the bacterial colonies on each plate were enumerated according to the manufacturer's instructions. The final concentration of bacterial levels on each experimental membrane was calculated.

### Statistical Analysis

Statistical analysis of the results was made using SPSS 12.0 statistical software (SPSS, Inc. Chicago, Ill.). One-way analysis of variance and paired t test were used. A P value less than 0.05 was considered statistically significant.

### Results

### Zinc Phosphate Mineralized Resolut Adapt LT (Copolymer)

Scanning electron microscopy analysis showed that the original shape and size of the membranes were not significantly changed after mineralization using either precipitation or microwave methods. Greater amounts of precipitate were observed in membranes mineralized using the microwave method (15 minutes) compared to those observed using the precipitation method (7 days). Using the precipitation method, the amount of precipitates increased with time (3 VS. 7 days).

Energy dispersive system spectra of mineralized films and precipitates showed the presence of mainly zinc and phosphate ions (FIG. 17). The presence of small amounts of other elements (Pt, K, and Ti) may be due to contamination from the sputter coating chamber.

Thermogravimetry analysis showed that mineralization by the microwave method for 15 minutes allowed the inclusion of significantly more zinc phosphate (average mineral weight, 21.39 wt percent) compared to the precipitation method for 7 days (average mineral weight, 18.35 wt percent).

### Zinc Phosphate Mineralization of BioMend Extend (Collagen)

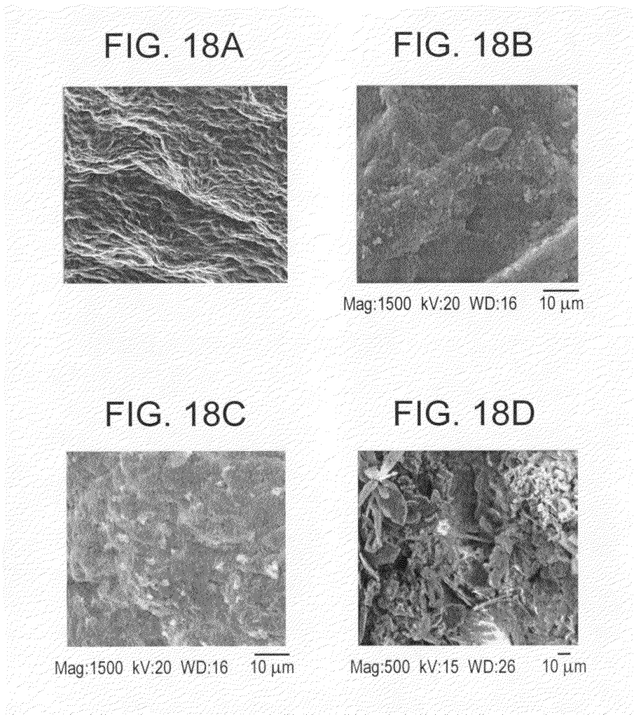

The size of the membrane increased by 2 times after mineralization using either precipitation or microwave methods. The texture of the membranes became more rigid and solid to the touch. The formation of crystals was observed with scanning electron microscopy (FIG. 18). Larger and greater amounts of crystals were observed on the membranes mineralized using the microwave method (FIG. 18D).

The energy dispersive system analysis showed the presence of mainly zinc and. phosphorus ions (identical to FIG. 17). Thermogravimetry analysis showed that the mineralization by the microwave method for 12-15 minutes allowed the inclusion of significantly more zinc phosphate (average mineral weight, 24.5 wt percent) compared to the precipitation method for 7 days (average mineral weight, 18.05 wt percent).

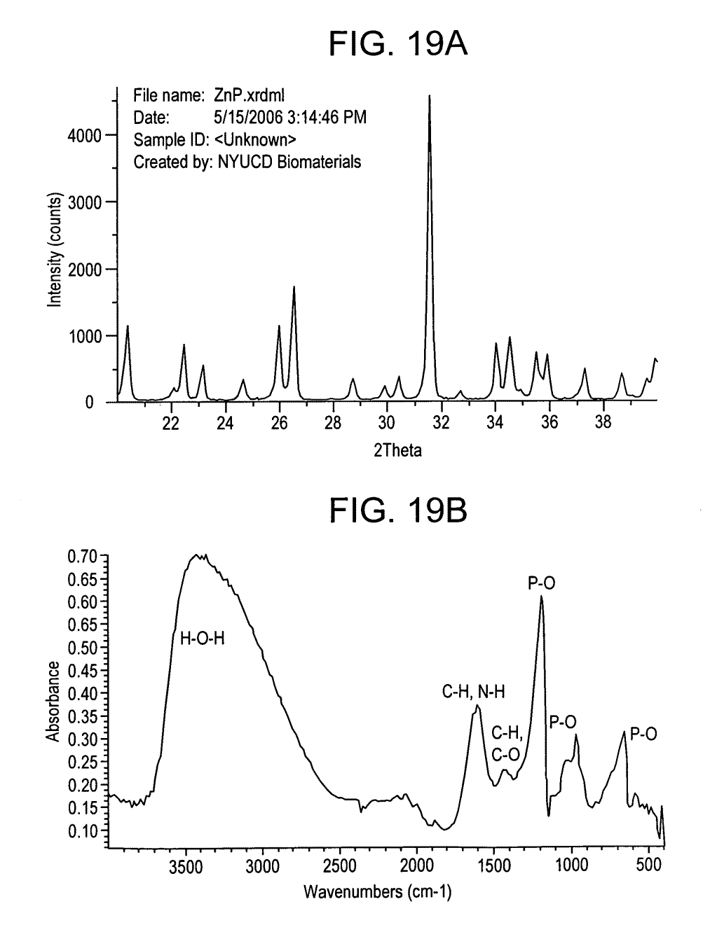

The x-ray diffraction analysis of material scraped from the surface of the mineralized membranes showed the presence of zinc phosphate (FIG. 19A).

Fourier transform infrared spectra of material scraped from the surface of the mineralized membranes showed absorption bands of the collagen (at 1500-1800 cm-′), phosphate (at 5001700 cm-′), and adsorbed water (at 2700-3600 cm-′) (FIG. 19B).

Inductive coupled plasma analysis showed that the amount of zinc and phosphate ions released in solution after incubation of zinc phosphate mineralized collagen and zinc phosphate mineralized copolymer membranes decreased with time from days 3 to 15. The maximum amount of zinc ions was released on day 3 in both zinc phosphate mineralized collagen (4.3±0.03) and copolymer membranes (3.8±0.014). The maximum amount of phosphate ions was also released on day 3 for both zinc phosphate mineralized collagen membranes (0.70±0.02) and zinc phosphate mineralized copolymer membranes (0.76±0.06). The zinc and phosphate ions continue to be released from the mineralized membranes on day 15.

### Effect of Zinc Phosphate Mineralized Membranes on Bacterial Colonization

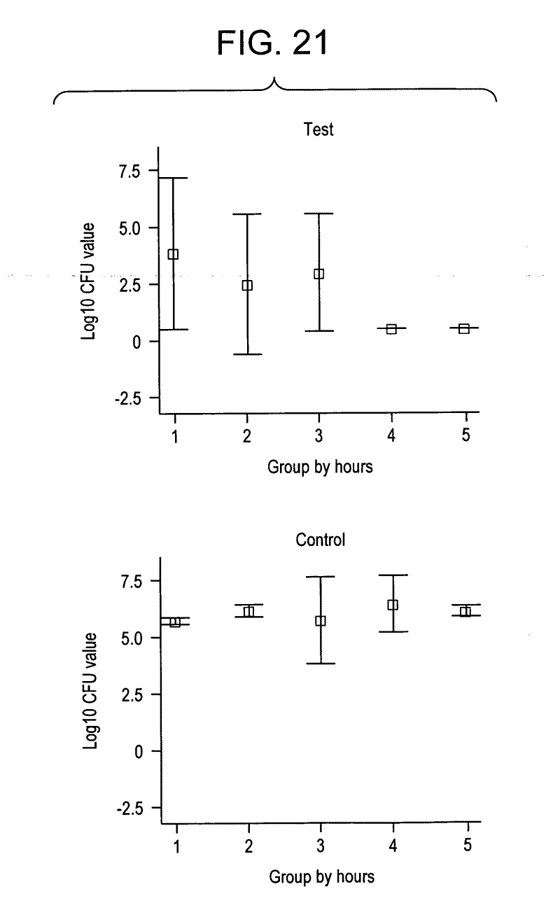

The colony forming units of the bacterial colonies on each plate were enumerated (FIG. 20). The mean colony forming unit values for nonmineralized collagen membranes (BioMend Extend, BC) and for zinc phosphate mineralized collagen membranes (BioMend Extend, BT) at 4, 8, 12, 24, and 48 hours after the inoculation are: BC: 5.655±0.608, 6.108±0.1353, 5.701±0.9524, 6.410±0.6303, and 6.087±0.1230; and BT: 3.810±1.6624, 2.451±1.4506, 2.956±1.2783, 0.500±0.000, and 0.500±0.000, respectively (FIG. 21).

The bacterial counts of BT and BC at different incubation times were compared and analyzed using the paired t test. Comparing the mean colony forming unit value for BT between 4 and 24 hours showed more than a 3-fold drop at 24 hours mean (3.520±1.8624 after 4 hours vs. 0.500±0.000 after 24 hours) in antibacterial effect (P<0.001). The difference in the overall mean colony forming unit value between BT and BC (0.500±0.000 for BT after 24 hours vs. 6.410±0.6303 for BC after 24 hours) was statistically significant (P<0.001).

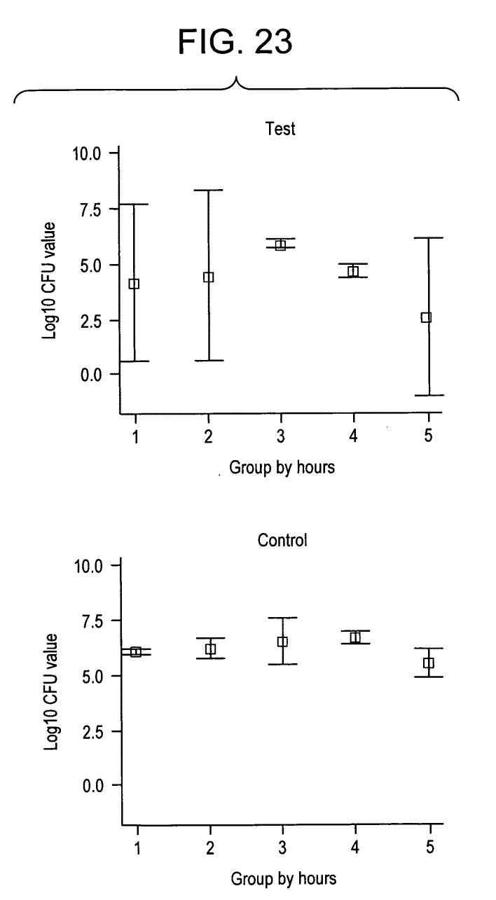

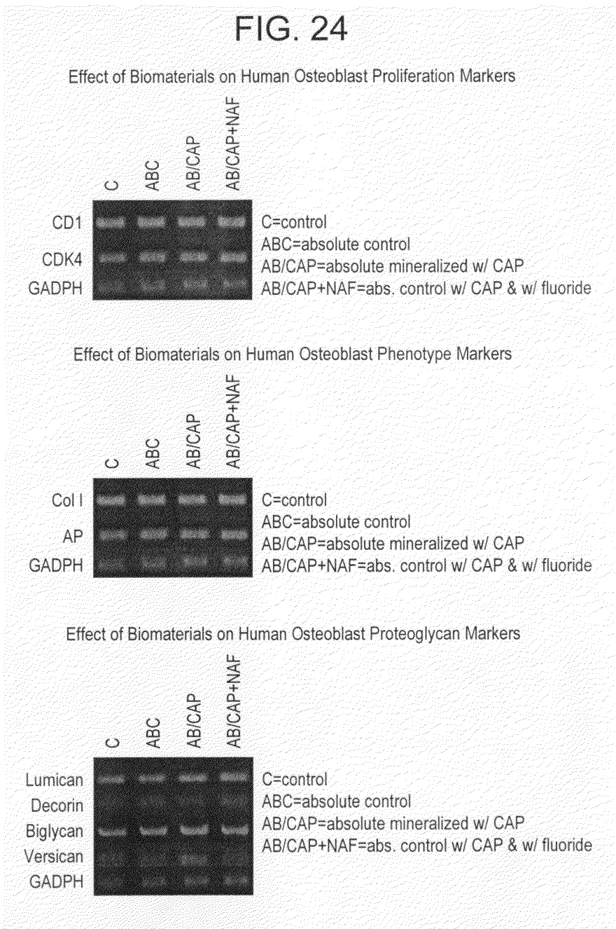

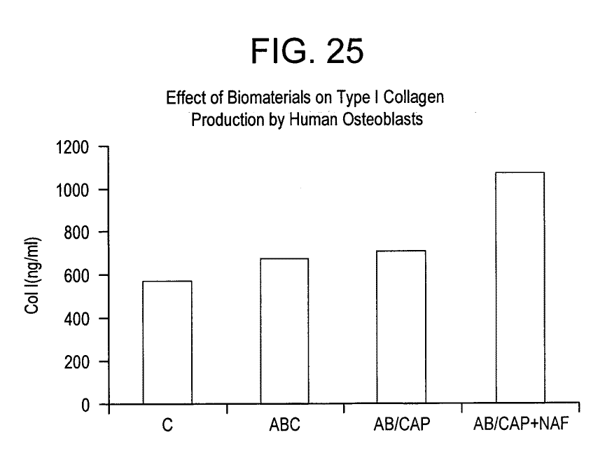

The antibacterial effects of nonmineralized (RC) and zinc phosphate mineralized Resolut Adapt L T (RT) were also evaluated at different incubation times (FIGS. 22 and 23). RT decreased statistically in the mean value after 48 hours. However, the difference in the mean colony forming unit value between RT and RC from 4 to 24 hours incubation time was not statistically significant.

The difference in the mean Log colony forming unit value between BC (5.943±0.54) and RC (6.259±0.43) was statistically significant (P=0.016). The difference in the mean Log₁₀ colony forming unit value between BT (2.432±1.74) and RT (4.520±1.67) was also statistically significant. Among the membranes tested, BT showed better antibacterial effect than RT. The nonmineralized membranes showed significantly higher counts of bacterial colonization compared to zinc phosphate mineralized membranes.

We claim:

1. A method for mineralizing a guided bone regeneration membrane comprising the steps of:
(a) providing a commercially available guided bone regeneration membrane;
(b) applying a mineralizing solution; and
(c) exposing the membrane to microwave radiation.

2. The method according to claim 1 further comprising:
(d) rinsing the membrane in a solution; and
(e) drying the membrane.

3. The method according to claim 1 wherein the mineralizing solution is a solution capable of supplying or delivering a mineral selected from the group consisting of calcium and zinc.

4. The method according to claim 1 wherein the exposing to microwave radiation step is performed for at least 10 minutes.

5. The method according to claim 1 wherein the exposing to microwave radiation step is performed for at least 12 minutes.

6. The method according to claim 1 wherein the exposing to microwave radiation step is performed for at least 15 minutes.

7. The method according to claim 1 wherein the mineral comprises at least 10% (weight percent) of the membrane at the conclusion of step (c).

8. The method according to claim 1 wherein the mineral comprises at least 12% (weight percent) of the membrane at the conclusion of step (c).

9. The method according to claim 1 wherein the mineral comprises at least 15% (weight percent) of the membrane at the conclusion of step (c).

10. The method according to claim 1 wherein the mineral comprises at least 20% (weight percent) of the membrane at the conclusion of step (c).

11. The method according to claim 1 wherein the membrane has a width of at least about 1.25 times its width as determined before the method was performed.

12. The method according to claim 1 wherein the membrane has a width of at least about 1.5 times its width as determined before the method was performed.

13. The method according to claim 1 wherein the membrane has a width of at least about 1.67 times its width as determined before the method was performed.

14. The method according to claim 1 wherein the membrane has a width of at least about, at least 1.75 its width as determined before the method was performed.

15. The method according to claim 1 wherein the membrane has a width of at least about two times its original width as determined before the method was performed.

16. A mineralized guided bone regeneration membrane.

17. A mineralized guided bone regeneration membrane according to claim 16 comprising one or more compound selected from the group consisting of poly-lactic acid (PLA), polylactic-glycolic acid (PLGA), collagen, e-PTFE and teflon.

18. A mineralized guided bone regeneration membrane according to claim 16 comprising one or more compound selected from the group consisting of PLA and PLGA.

19. A mineralized guided bone regeneration membrane made by the method according to claim 1.

20. A mineralized guided bone regeneration membrane according to claim 16 comprising a mineral selected from the group consisting of calcium and zinc.

21. A mineralized guided bone regeneration membrane according to claim 20 wherein the mineral comprises at least 5% (weight percent) of the membrane.

22. A mineralized guided bone regeneration membrane according to claim 20 wherein the mineral comprises at least 10% (weight percent) of the membrane.

23. A mineralized guided bone regeneration membrane according to claim 20 wherein the mineral comprises at least 12% (weight percent) of the membrane.

24. A mineralized guided bone regeneration membrane according to claim 20 wherein the mineral comprises at least 15% (weight percent) of the membrane.

25. A mineralized guided bone regeneration membrane according to claim 20 wherein the mineral comprises at least 18% (weight percent) of the membrane.

26. A mineralized guided bone regeneration membrane according to claim 20 wherein the mineral comprises at least 20% (weight percent) of the membrane.

27. A mineralized guided bone regeneration membrane according to claim 20 wherein the mineral comprises or at least 25% (weight percent) of the membrane.

28. A mineralized guided bone regeneration membrane according to claim 20 wherein more than one mineral is present.

29. A mineralized guided bone regeneration membrane according to claim 16 having a width of at least about 1.25 times its original width as determined before the mineralizing process was performed.

30. A mineralized guided bone regeneration membrane according to claim 16 having a width of at least about at least 1.5 times its original width as determined before the mineralizing process was performed.

31. A mineralized guided bone regeneration membrane according to claim 16 having a width of at least about 1.67 times its original width as determined before the mineralizing process was performed.

32. A mineralized guided bone regeneration membrane according to claim 16 having a width of at least about 1.75 times its original width as determined before the mineralizing process was performed.

33. A mineralized guided bone regeneration membrane according to claim 16 having a width of at least about two times its original width as determined before the mineralizing process was performed.

34. The guided bone regeneration membrane according to claim 16 biologically active to allow attachment, proliferation, migration or phenotypic expression of bone cells.

35. The guided bone regeneration membrane according to claim 16 biologically active to promote increased collagen production.

36. The guided bone regeneration membrane according to claim 16 biologically active to inhibit bacterial colonization, bacterial infection or inflammation.

37. A method for promoting or enhancing bone regeneration comprising providing a mineralized guided bone regeneration membrane according to claim 16.

38. A method for promoting or enhancing bone regeneration comprising providing a mineralized guided bone regeneration membrane according to claim 19.

39. A method for inhibiting bacterial colonization, for inhibiting bacterial infection or for reducing inflammation comprising providing a mineralized guided bone regeneration membrane according to claim 16.

40. A method for inhibiting bacterial colonization, for inhibiting bacterial infection or for reducing inflammation comprising providing a mineralized guided bone regeneration membrane according to claim 19.
